(19) United States
(12) Patent Application Publication
(10) Pub. No.: US 2023/0042140 A1
(43) Pub. Date: Feb. 9, 2023

(54) OCCLUSION DETECTION IN BODY CAVITIES

(71) Applicant: Navix International Limited, Road Town, Tortola (VG)

(72) Inventor: Shlomo BEN-HAIM, Milan (IT)

(73) Assignee: Navix International Limited, Road Town, Tortola (VG)

(21) Appl. No.: 17/789,802

(22) PCT Filed: Dec. 30, 2020
(86) PCT No.: PCT/IB2020/062543

§ 371 Date: Jun. 29, 2022

Related U.S. Application Data
(60) Provisional application No. 63/059,203, filed on Jul. 31, 2020.
(60) Provisional application No. 63/043,156, filed on Jun. 24, 2020.
(60) Provisional application No. 62/990,004, filed on Mar. 16, 2020.
(60) Provisional application No. 62/960,023, filed on Jan. 12, 2020.
(60) Provisional application No. 62/956,249, filed on Jan. 1, 2020.

(51) Int. Cl. A61B 17/00; A61B 17/12

(52) U.S. Cl. CPC A61B 17/0057; A61B 17/12022; A61B 2017/00243

(57) ABSTRACT

Degree of occlusion is monitored for an occlusive device configured to occlude passage of fluid between two compartments in a lumenal space of a body of a patient. In some embodiments, changes in an electrical signal measured from the body of the patient are induced by perturbing the fluid; for example, by “tagging” a portion of fluid with a perturbation of temperature and/or composition. The degree of occlusion is estimated based on the measured changes. The electrical signal changes may be indicative of fluid movements redistributing the perturbed fluid among the two compartments; for example, by diffusion, mixing, and/or jetting of fluid.

### RELATED APPLICATIONS

This application claims the benefit of priority of:
U.S. Provisional Pat. Application No. 62/956,249, filed on Jan. 1, 2020;
U.S. Provisional Pat. Application No. 62/960,023, filed on Jan. 12, 2020;
U.S. Provisional Pat. Application No. 62/990,004, filed on Mar. 16, 2020;
U.S. Provisional Pat. Application No. 63/043,156, filed on Jun. 24, 2020; and
U.S. Provisional Pat. Application No. 63/059,203, filed on Jul. 31, 2020;
the contents of which are incorporated herein by reference in their entirety.

### FIELD AND BACKGROUND OF THE INVENTION

The present invention, in some embodiments thereof, relates to the field of placement of intrabody devices within body cavities, and more particularly, to monitoring of the placement of intrabody devices, optionally including implantable devices.

Several medical procedures in cardiology and other medical fields comprise the use of intrabody devices such as catheter probes to reach tissue targeted for diagnosis and/or treatment while minimizing procedure invasiveness. Treatments include occlusive devices which deploy to obstruct a normal or abnormal aperture such as an opening in a tissue wall (e.g., an atrial or ventricular septal defect in a heart, or a patent foramen ovale), an ostium of a lumenal structure (e.g., such an ostium of a left atrial appendage), or a body of a tubular lumen (e.g., a fallopian tube or a vas deferens).

Occlusive device deployment may be monitored by imaging methods such as fluoroscopy and ultrasound.

### SUMMARY OF THE INVENTION

According to an aspect of some embodiments of the present disclosure, there is provided a method of determining a state of occlusion resulting from an occlusive device positioned between two compartments of a lumenal space of a body of a patient, the method including: measuring changes in an electrical signal measured from the body of the patient, wherein the electrical signal changes are indicative of changes in a fluid within the lumenal space elicited by a perturbation of the fluid; and estimating the state of occlusion based on the measured electrical signal changes.

Also according to this aspect of some embodiments of the present disclosure, there is provided a system configured to determine a state of occlusion resulting from an occlusive device positioned between two compartments of a lumenal space of a body of a patient, the system including a computer processor and memory containing instructions which instruct the computer processor to: measure changes in an electrical signal measured from the body of the patient, wherein the electrical signal changes are indicative of changes in a fluid within the lumenal space elicited by a perturbation of the fluid; and estimate the state of occlusion based on the measured electrical signal changes.

According to some embodiments of the present disclosure, the processor estimates the state of occlusion by identifying a decay rate in the electrical signal induced by the perturbation.

According to some embodiments of the present disclosure, the processor estimates the state of occlusion by comparing the decay rate to a predetermined threshold.

According to some embodiments of the present disclosure, the processor estimates the state of occlusion by classifying the changes in the electrical signal to estimate the state of occlusion.

According to some embodiments of the present disclosure, the processor classifies the changes by comparing an identified decay rate in the electrical signal to a predetermined threshold, and classifying the changes based on the comparison.

According to some embodiments of the present disclosure, the processor estimates the state of occlusion as a satisfactory state of occlusion, based on the identified decay rate being indicative of slower decay than indicated by the predetermined threshold.

According to some embodiments of the present disclosure, the processor is further configured to determine a location of a leak of the fluid between the two compartments.

According to some embodiments of the present disclosure, the system further includes a display which the processor controls to present an indication of the determined state of occlusion.

According to some embodiments of the present disclosure, the changes in the fluid comprise changes in an electrical property of the fluid.

According to some embodiments of the present disclosure, the method further includes causing the perturbation.

According to some embodiments of the present disclosure, the perturbation of the fluid and the changes in the fluid both comprise adjustment of the fluid to a warmer or cooler temperature.

According to some embodiments of the present disclosure, causing the perturbation includes changing a chemical composition of the fluid.

According to some embodiments of the present disclosure, the perturbation includes injecting a dielectric contrast agent into the fluid.

According to some embodiments of the present disclosure, the injecting includes pressing the dielectric contrast agent across a membrane of the occlusive device.

According to some embodiments of the present disclosure, causing the perturbation includes generating bubbles within the fluid.

According to some embodiments of the present disclosure, the estimating includes identifying a decay rate in the electrical signal induced by the perturbation.

According to some embodiments of the present disclosure, the estimating further includes comparing the decay rate to a predetermined threshold.

According to some embodiments of the present disclosure, the estimating includes classifying the changes in the electrical signal, using a computer processor, to estimate the state of occlusion.

According to some embodiments of the present disclosure, the classifying includes comparing an identified decay rate in the electrical signal to a predetermined threshold, and classifying the changes based on the comparison.

According to some embodiments of the present disclosure, the method includes estimating the state of occlusion to be a satisfactory state of occlusion, based on the identified decay rate being indicative of slower decay than indicated by the predetermined threshold.

According to some embodiments of the present disclosure, the changes in the electrical signal are indicative of movement of the fluid following the perturbation.

According to some embodiments of the present disclosure, the movement of the fluid includes a changing distribution within the fluid of a perturbed portion of the fluid.

According to some embodiments of the present disclosure, the changing distribution includes at least one of the group consisting of a reduction in amount and an increase in amount of the perturbed portion of the fluid in at least one of the two compartments.

According to some embodiments of the present disclosure, the changing distribution includes heartbeat-synchronized changes in the distribution of the perturbed portion of the fluid.

According to some embodiments of the present disclosure, the changing distribution includes changes in average concentration of the perturbed portion of the fluid over the course of a plurality of heartbeats.

According to some embodiments of the present disclosure, the estimating includes determining a location of a leak of the fluid between the two compartments.

According to some embodiments of the present disclosure, the measuring includes measuring from a plurality of electrodes, and the determining a location includes detection of differences in measurements among the plurality of electrodes.

According to some embodiments of the present disclosure, the two compartments are in a heart of a patient.

According to some embodiments of the present disclosure, one of the two compartments includes a left atrial appendage and the other includes a remaining lumenal portion of the left atrium.

According to some embodiments of the present disclosure, one of the two compartments includes a left atrium or a left ventricle; and the other compartment includes a right atrium or right ventricle, respectively.

According to some embodiments of the present disclosure, the method further includes controlling a display to present an indication of the determined state of occlusion.

According to some embodiments of the present disclosure, the method further includes presenting to a user an indication of the determined state of occlusion.

According to some embodiments of the present disclosure, the electrical signal is measured as electrical impedance.

According to some embodiments of the present disclosure, the electrical impedance is measured as a ratio between a voltage and a current, wherein the voltage is a voltage measured between an electrode portion of the occlusive device and a reference electrode; and the current is an electrical current injected to the electrode portion of the occlusive device.

According to some embodiments of the present disclosure, the measuring is performed using an electrode portion of the occlusive device.

According to some embodiments of the present disclosure, the electrical signal includes a voltage measured between the electrode portion of the occlusive device, and a reference electrode.

According to some embodiments of the present disclosure, the reference electrode is attached to the body surface of the patient.

According to some embodiments of the present disclosure, the electrical signal indicates a property of an electrical current injected to the electrode portion of the occlusive device.

According to some embodiments of the present disclosure, the lumenal space includes lumenal portions of a heart, and the changes in the electrical signal are driven at least in part by beating of the heart.

According to an aspect of some embodiments of the present disclosure, there is provided a method of providing an estimated state of occlusion resulting from an occlusive device positioned between two compartments of a lumenal space of a body of a patient, the method including: accessing electrical measurements measured using an electrode portion of the occlusive device; estimating the state of occlusion based on the accessed electrical measurements; and providing an indication of the estimated state of occlusion.

According to some embodiments of the present disclosure, the method further includes presenting the provided indication.

According to some embodiments of the present disclosure, the accessed electrical measurements comprise measurements made during a period following injection of a dielectric contrast agent within at least one of the two compartments.

According to some embodiments of the present disclosure, the occlusive device has a proximal side attached to a delivery device, and a distal side; and the dielectric contrast agent is injected to the compartment on the distal side of the occlusive device.

According to some embodiments of the present disclosure, the dielectric contrast agent is pressed across a membrane of the occlusive device into the compartment on the distal side of the occlusive device.

According to some embodiments of the present disclosure, the state of occlusion is estimated based on a variation of the electrical measurements over time.

According to some embodiments of the present disclosure, the estimating includes classifying the electrical measurements, using a computer processor, to obtain the estimated state of occlusion.

According to some embodiments of the present disclosure, the classifying includes identifying a rate at which changes in the electrical measurements return toward their values pre-injection.

According to some embodiments of the present disclosure, the classifying further includes comparing the identified rate to a predetermined threshold, and classifying the electrical measurements based on the comparison.

According to some embodiments of the present disclosure, the method includes estimating the state of occlusion to be a satisfactory state of occlusion, based on the identified decay rate being indicative of slower decay than indicated by the predetermined threshold.

According to some embodiments of the present disclosure, the lumenal space includes lumenal portions of a heart, and the state of occlusion is estimated based on changes in the electrical measurements driven at least in part by beating of the heart.

According to some embodiments of the present disclosure, the occlusive device occludes an opening between a left atrial appendage and a remaining lumen of a heart left atrium.

According to some embodiments of the present disclosure, the occlusive device occludes an opening between a left atrium and a right atrium of a heart.

According to some embodiments of the present disclosure, the occlusive device occludes an opening between a left ventricle and right ventricle of a heart.

According to some embodiments of the present disclosure, the electrical measurements include measurements of voltage between the electrode portion of the occlusive device, and a reference electrode.

According to some embodiments of the present disclosure, the reference electrode is attached to a body surface of the patient.

According to some embodiments of the present disclosure, the electrical measurements include measurements of electrical current injected to the electrode portion of the occlusive device.

According to some embodiments of the present disclosure, the electrical measurements include impedance measurements.

According to some embodiments of the present disclosure, the impedance is a ratio between a voltage and current, wherein the voltage is a voltage measured between the electrode portion of the occlusive device and a reference electrode; and the current is an electrical current injected to the electrode portion of the occlusive device.

According to an aspect of some embodiments of the present disclosure, there is provided an apparatus for presenting an indication of a state of occlusion resulting from an occlusive device positioned between two compartments of a lumenal space of a body of a patient, the apparatus including a processor configured to: access electrical measurements measured using an electrode portion of the occlusive device; estimate a state of occlusion based on the accessed electrical measurements; and present an indication of the estimated state of occlusion.

According to some embodiments of the present disclosure, the processor is further configured to control a display to present the indication of the estimated state of occlusion.

According to some embodiments of the present disclosure, the apparatus further includes the display.

According to some embodiments of the present disclosure, the apparatus further includes an interface configured to provide the processor with a predetermined signal when a dielectric contrast agent is being injected on at least one side of the occlusive device, and the processor is configured to use the predetermined signal in estimating the state of occlusion.

According to some embodiments of the present disclosure, the processor is configured to estimate the state of occlusion based on a change of the electrical measurements over time.

According to some embodiments of the present disclosure, the processor is configured to estimate the state of occlusion based on a change of the electrical measurements over time and the change of the electrical measurements begins when the predetermined signal is received.

According to some embodiments of the present disclosure, the processor is configured to classify the electrical measurements to obtain the estimated state of occlusion.

According to some embodiments of the present disclosure, processor is configured to classify the electrical measurements according to a time duration beginning upon receiving the predetermined signal, and ending upon returning of the electrical measurements to their values pre-injection.

According to some embodiments of the present disclosure, the processor is configured to compare the time duration to a predetermined threshold, and classify the electrical measurements based on the comparison.

According to some embodiments of the present disclosure, the processor is configured to: classify the electrical measurements to obtain the estimated state of occlusion according to a rate of decay toward a baseline value of the measurements estimated from measurements obtained after receiving the predetermined signal; compare the rate of decay to a predetermined threshold; and control the display to present an indication to the occlusion being satisfactory when the rate of decay is slower than the predetermined threshold.

According to some embodiments of the present disclosure, the apparatus further includes a voltmeter configured to measure a voltage between the electrode portion of the occlusive device and a reference electrode, and the electrical measurements include voltage measurement made by the voltmeter.

According to some embodiments of the present disclosure, the apparatus further includes an electrical current source, configured to inject electrical current to the electrode portion of the occlusive device, and the electrical measurements include measurements of voltage made when current is injected from the electrical current source to the electrode portion of the occlusive device.

According to some embodiments of the present disclosure, the electrical current is an alternating current of a predetermined frequency, and the voltage measurements are measurements of voltage oscillating at the predetermined frequency.

According to some embodiments of the present disclosure, the apparatus further includes an amperemeter configured to measure electrical current going through the electrode portion of the occlusive device, and the electrical measurements include current measurements made by the amperemeter when current is injected from the electrical current source to the electrode portion of the occlusive device.

According to some embodiments of the present disclosure, the electrical current is an alternating current of a predetermined frequency, the amperemeter is configured to measure currents oscillating at the predetermined frequency, and the electrical measurements include current measurements made by the amperemeter at the predetermined frequency.

According to an aspect of some embodiments of the present disclosure, there is provided a method of estimating a state of occlusion resulting from an occlusive device positioned between two compartments of a lumenal space of a body of a patient, the method including: accessing electrical measurements measured using an electrode portion of the occlusive device; and estimating the state of occlusion based on a variation of the electrical measurements over time.

According to some embodiments of the present disclosure, the method further includes providing an indication of the estimated state of occlusion.

According to some embodiments of the present disclosure, the estimating includes classifying the electrical measurements, using a computer processor, to estimate the state of occlusion.

According to some embodiments of the present disclosure, the classifying includes identifying a rate at which changes in the electrical measurements return toward their values pre-injection.

According to some embodiments of the present disclosure, the classifying further includes comparing the identified rate to a predetermined threshold, and classifying the electrical measurements based on the comparison.

According to some embodiments of the present disclosure, the occlusive device occludes an opening between a left atrial appendage and a remaining lumen of a heart left atrium.

According to some embodiments of the present disclosure, the electrical measurements include measurements of voltage between the electrode portion of the occlusive device, and a reference electrode.

According to some embodiments of the present disclosure, the reference electrode is attached to a body surface of the patient.

According to an aspect of some embodiments of the present disclosure, there is provided a method of estimating a state of occlusion resulting from an occlusive device positioned between two compartments of a lumenal space of a body of a patient, the method including: accessing electrical measurements measured using an electrode portion of the occlusive device; estimating the state of occlusion based on the electrical measurements; wherein the estimating includes classifying, using a computer processor, the electrical measurements to obtain the estimated state of occlusion.

According to an aspect of some embodiments of the present disclosure, there is provided a method of determining a state of occlusion between two compartments of a lumenal space of a body of a patient, the method including: measuring an electrical signal from the body of the patient indicative of perturbed fluid locations within the lumenal space; and estimating the state of occlusion based on the measured electrical signal.

According to some embodiments of the present disclosure, the occlusion is caused by an occlusive device.

According to some embodiments of the present disclosure, the occlusive device is positioned between the two compartments.

According to some embodiments of the present disclosure, the perturbed fluid has an electrically distinct characteristic indicated in the electrical signal.

According to some embodiments of the present disclosure, the method further includes perturbing the perturbed fluid.

According to some embodiments of the present disclosure, the perturbed fluid is warmed or cooled relative to a pre-perturbation temperature.

According to some embodiments of the present disclosure, the perturbed fluid has a perturbed chemical composition.

According to some embodiments of the present disclosure, the perturbing includes injecting a dielectric contrast agent into the fluid.

According to some embodiments of the present disclosure, the injecting includes pressing the dielectric contrast agent through a membrane of the occlusive device.

According to some embodiments of the present disclosure, the perturbing includes generating bubbles within the fluid.

According to some embodiments of the present disclosure, the estimating includes identifying a rate of change in the electrical signal.

According to some embodiments of the present disclosure, the estimating further includes comparing the rate of change to a predetermined threshold.

According to some embodiments of the present disclosure, the estimating includes classifying measurements of the electrical signal, using a computer processor, to estimate the state of occlusion.

According to some embodiments of the present disclosure, the rate of change is indicative of movement of the fluid following the perturbation.

According to some embodiments of the present disclosure, the movement includes at one or both of a reduction in amount and an increase in amount of the perturbed fluid in at least one of the two compartments.

According to some embodiments of the present disclosure, determination of the rate of change includes removal of a heartbeat-synchronized signal from the electrical signal.

According to some embodiments of the present disclosure, determination of the rate of change includes removal of a breathing-synchronized signal from the electrical signal.

According to some embodiments of the present disclosure, the removal of a breathing-synchronized signal from the electrical signal includes two passes of estimating the breathing-synchronized signal.

According to some embodiments of the present disclosure, the removal of a breathing-synchronized signal from the electrical signal includes two passes of estimating the rate of change in the electrical signal indicative of perturbed fluid locations within the lumenal space.

According to an aspect of some embodiments of the present disclosure, there is provided a method of determining a degree of occlusion obtained by deployment of an occlusive device to divide a body lumen, the method including: accessing electrical impedance measurements measured using a portion of the occlusive device as an electrode during deployment of the device; estimating a degree of occlusion based on the measured electrical impedance; and presenting an indication of the estimated degree of occlusion.

According to some embodiments of the present disclosure, the estimating includes classifying, using a computer processor, the electrical impedance measurements to obtain an estimated degree of occlusion.

According to some embodiments of the present disclosure, the estimating includes analyzing a period of unstable electrical impedance measurements recorded after deployment.

According to some embodiments of the present disclosure, a period for assessing the electrical impedance measurement stability includes at least five seconds after an initial drop in electrical impedance during deployment of the occlusive device.

According to some embodiments of the present disclosure, the at least five seconds begin after a partial rebound in electrical impedance during deployment of the occlusive device.

According to some embodiments of the present disclosure, the classifying includes identifying an initial drop in impedance followed by a partial rebound in impedance as indicating deployment of the occlusive device within a confined location.

According to some embodiments of the present disclosure, a dielectric contrast agent is injected on at least one side of the occlusive device; and the degree of occlusion is estimated based on a time course of the measured electrical impedance as the dielectric contrast agent redistributes.

According to some embodiments of the present disclosure, the occlusive device occludes an opening between a left atrial appendage and a remaining lumen of a heart left atrium.

According to an aspect of some embodiments of the present disclosure, there is provided a method of determining a degree of occlusion obtained by deployment of an occlusive device to divide a body lumen, the method including: accessing electrical impedance measurements measured using body surface electrodes; estimating a degree of occlusion based on the measured electrical impedance; and presenting an indication of the estimated degree of occlusion.

According to some embodiments of the present disclosure, the apparatus is provided together with an electrical interconnection device configured to receive signals from an occlusive device.

According to an aspect of some embodiments of the present disclosure, there is provided a kit comprising an occlusive device, and an electrical conductor configured to attach to the occlusive device, and sized to transmit an electrical signal from the occlusive device to an electrical interface, while the occlusive device is positioned within a body lumen.

According to an aspect of some embodiments of the present disclosure, there is provided a kit for adapting an occlusive device to use as an electrode, the kit comprising an elongated electrical conductor configured to attach to the occlusive device; sized to transmit an electrical signal from an electrical connection it makes with the occlusive device to an electrical interface, while the occlusive device is positioned within a body lumen; and configured to release from the occlusive device and be withdrawn from the body lumen while the occlusive device remains inside.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user’s computer, partly on the user’s computer, as a stand-alone software package, partly on the user’s computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWING(S)

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a flowchart schematically representing a method of monitoring a state of occlusion between two fluid-filled compartments of a body lumen, according to some embodiments of the present disclosure.

FIG. 2 shows example time courses of voltage measurements made using an LAA occlusive device as an electrode referenced (grounded) to a body surface pad electrode upon injection of Iodixanol solution, comparing leaking and closed deployments of an LAAO device, according to some embodiments of the present disclosure.

FIG. 3 shows example time courses of voltage measurements made using an LAA occlusive device as an electrode referenced (grounded) to a body surface pad electrode upon injection of saline solution, comparing leaking and closed deployments of an LAAO device, according to some embodiments of the present disclosure;

FIG. 4 summarizes experimental results of closed occlusion vs. leaking occlusion time-course measurements for a population of LAAO closure trials (in pig hearts), according to some embodiments of the present disclosure; and

FIG. 5 schematically represents an apparatus for estimating a state of occlusion of an occlusive device, according to some embodiments of the present disclosure;

FIG. 6 is another flowchart schematically representing a method of monitoring state of occlusion between two fluid-filled compartments of a body lumen, according to some embodiments of the present disclosure;

FIGS. 7A-7B schematically illustrate a method of isolating impedance changes due to dilution from a cardiac recording, according to some embodiments of the present disclosure; and

FIGS. 8A-8B graph sealing test experimental results in dog heart, according to some embodiments of the present disclosure.

FIG. 9A is a flowchart schematically representing a method of verifying implant positioning, according to some embodiments of the present disclosure;

FIGS. 9B-9C are schematic graphs of impedance over time for stable and unstable closure by an LAAO device, according to some embodiments of the present disclosure;

FIG. 9D schematically represents an electrical measurement configuration related to the measurements of FIGS. 9B-9C, according to some embodiments of the present disclosure;

### DESCRIPTION OF SPECIFIC EMBODIMENTS OF THE INVENTION

The present invention, in some embodiments thereof, relates to the field of placement of intrabody devices within body cavities, and more particularly, to monitoring of the placement of intrabody devices, optionally including implantable devices.

### Overview

The present invention, in some embodiments thereof, relates to a method of estimating and/or monitoring a state of occlusion by an occlusive device that occludes a passageway between two body lumens of a patient. Occluding a passageway between two lumens may be referred to herein as dividing a lumen.

The two lumens may be two parts of a pre-defined lumenal space that is divided to two lumens (also referred to herein as “compartments”) by the occlusive device. The lumens may be in a heart of the patient. For example, the two lumens optionally comprise a left atrium and a right atrium of a heart (with occlusion, e.g., of a foramen ovale); a right ventricle and a left ventricle of a heart (with occlusion, e.g., of a ventricular septal defect); or a left atrium of a heart and an appendage of said left atrium (with occlusion, e.g., of an orifice of the left atrial appendage).

In embodiments, the method may include measuring changes in an electrical signal measured using a device positioned within the body of the patient and operated as a sensing electrode; the electrical signal changes following a perturbation of fluid in one of the compartments. The perturbation affects the electrical signal, e.g., by modifying an electrical property of fluid in one of the lumens. The electrical signal comprises, for example, a measured voltage, current, and/or impedance. Measurements optionally comprise, for example, measurements of amplitude and/or phase. Perturbations that may affect electrical properties comprise, for example, warming and or cooling of the fluid, and/or the creation of microbubbles, e.g. via ultrasonic vibration. In some embodiments, another sensor type is used, appropriate to a different perturbation and/or a different property modified by the perturbation; for example, a temperature sensor to measure injection of warmed or cooled saline, or a sound sensor configured to measure changes in sound propagation rate.

The electrical properties indicated by the electrical signal may be of fluid in the same lumen that was perturbed, or in the other one. In some embodiments, fluid on both lumens is perturbed, to different degrees. The fluid portion that is “perturbed” is the fluid portion that exhibits a property change (e.g., an electrically measurable property change) as a result of the perturbation. This portion can dilute as it redistributes. In some embodiments, the perturbation decays with time. The decay rate is generally slower in sealed lumens than in open lumens, because when the lumen is open it may exchange fluid with the other side of the opening, and the perturbation decays faster than if no fluid exchange (or less fluid change) takes place. The decay rate may be defined, for example, as time it takes for the entire perturbation to disappear (and the parameter returning to its pre-perturbation value), time of decay of the perturbation to a certain portion of its initial value (e.g., half), the percentage of perturbation decay occurring at some predetermined time period (e.g., 1 second, 1 heartbeat, etc.), or the like. The decay rate of the change in the electrical signal and/or the change in the dielectric properties may be indicative of the decay rate of the perturbation, and thus all these decays may be used herein interchangeably.

In some embodiments, estimating the state of occlusion comprises estimating a decay rate for the perturbation, and estimating the state of occlusion based on the estimate of the decay rate. For example, the decay rate may be compared to a threshold, e.g., a threshold that was determined empirically, and if the decay rate is slower than the threshold, this may lead to determination that the occlusion is satisfactory. Optionally, if the decay rate is faster than the threshold, the occlusion is unsatisfactory, and the occlusion should be improved, e.g., by changing a position and/or orientation of the occlusive device in the passageway to be occluded.

In some embodiments, the method may include causing the perturbation. Alternatively, the perturbation is caused before the method begins. For example, the perturbation may be caused by a physician (e.g., by injecting a fluid to one of the lumens), and the measurement and determination may be carried out by a system configured for measuring electrical signals from the body of the patient, and analyzing them according to the teachings provided herein.

Examples of perturbations include heating or cooling the fluid (either of which adjusts the temperature of the fluid), changing a chemical composition of the fluid, and changing an electrical conductivity of the fluid. Heating or cooling may be achieved, for example by injecting hot (or cold, as the case may be) blood or saline. Additionally or alternatively, heating comprises injection of energy to the fluid, e.g., by conductive and/or radiative heating. Heating or cooling may optionally comprise operation of a Peltier device. Electrical conductivity and/or chemical composition is perturbed, for example, by injecting a dielectric contrast agent, such as a saline or iodine containing material with conductivity different than that of the fluid. In another example, electrical conductivity may be changed by cavitation, e.g., generating microbubbles in the fluid ultrasonically.

In some embodiments, the method includes accessing electrical measurements made by an electrode portion of the occlusive device. The electrical measurements may be of the above-mentioned electrical signal; thus, the measurements may be indicative to the perturbation and its decay.

Herein, an electrode portion of a device is a portion of the device that is used as an electrode. It may be, for example, a conductive element with a non-electrode function in the device, such as a strut, arm, mesh, connector, hub, slider, or other element. Optionally, the electrode portion comprises an element added on to the device, e.g., applied to a device surface or otherwise attached. Such portion may be electrically conductive, and connected to an electrical field generator and/or to a meter. Optionally, the meter is configured to measure potential difference (i.e., voltage) between the electrode portion of the device and a reference electrode. Optionally or additionally, the meter may be configured to measure electrical current injected to the electrode portion. Optionally, the electrical measurements are electrical measurements made by the above-mentioned meter. In some embodiments, the electrical field may be generated by a field generator via an electrode portion of the device, e.g., the occlusive device. In some embodiments, the electrical field is alternating at a given frequency, and the meter is configured to measure the voltage and/or the current at the same given frequency. The measurements may be accessed, e.g., by a processor, by accessing digital data storage. The digital data storage may store data obtained before the method began, or may be accessed immediately after being stored, e.g., during or immediately after deployment of the occlusive device.

Based on the accessed electrical measurements, a state of occlusion may be estimated. Optionally, an indication of the estimated state of occlusion is provided for presentation. The presentation may be, for example, by presenting on a display an icon, numerical value, graph, text, or any other visual or audible indication of the estimated state of occlusion.

In some embodiments, the electrical measurements are measured following the perturbation. For example, following injection of a dielectric contrast agent on at least one side of the occlusive device. In the following passages, injection of dielectric contrast agents is described as examples. The descriptions apply also, changed as necessary for the use of different perturbation sources and/or sensors, to other perturbations; for example, thermal changes and/or the introduction of cavitations (bubbles).

In some embodiments, the occlusive device may have two sides. Upon being deployed to occlude an opening between two compartments, one side of the occlusive device faces one compartment, and another side of the occlusive device faces the other compartment. When the device is delivered to the opening on a catheter, the two sides of the device may be referred to as proximal and distal, with the proximal side being closer to a connection point between the catheter and the device, and the distal side being further away from that connection point. The catheter used for delivering the occlusive device is used, in some embodiments, for injecting a contrast agent from the proximal side of the occlusive device, optionally to the distal side thereof. It is sometimes preferred to have the contrast agent begin in the distal lumen; e.g., because the distal lumen has no exits for fluid other than back across the opening which the occlusive device is configured to close.

Accordingly, in some embodiments, the dielectric contrast agent is pressed across a membrane, port, or other permeable or flow-permitting structure of the occlusive device into the distal lumen. In some embodiments, the dielectric contrast agent is injected through an injection needle inserted to the distal lumen through the occlusive device. In some embodiments, the injection is done by a separate catheter. Optionally, the separate catheter and/or a tool passed therethrough is entered into to the distal lumen, preferably not through the passageway to be occluded, and injects the contrast agent directly into the distal lumen.

The dielectric contrast agent may be any solution with dielectric properties distinct from the dielectric properties of the fluid (e.g., blood) that normally fills the compartments into which the body lumen is to be divided. The difference should be large enough so that measurements sensitive to the concentration of the dielectric contrast agent allow monitoring how that concentration changes over time after injection.

Dielectric contrast agent may be or include, for example: a liquid having temperature different from the temperature of the fluid (e.g., blood) that normally fills the lumens, a passageway between them is to be occluded. The temperature difference should be large enough so that the electrical measurements allow monitoring how the concentration of the dielectric contrast agent changes over time after being injected to one of the lumens. Examples of such liquids include, ice cold saline, warm saline, cold blood, and warm blood. Additionally or alternatively, the dielectric contrast agent may have electrical conductivity that is different from that of the fluid (e.g., blood) that normally fills the lumens. The difference in electrical conductivity is preferably large enough to allow sensing changes in concentration of the dielectric contrast agent by following the dielectric measurements. Examples of such dielectric contrast agents include hypertonic saline, hypotonic saline, and iodine containing solution (e.g. Iodixanol).

As the dielectric contrast agent influences the electrical environment sensed by the electrode portion of the occlusive device, the electrical readings change. The contrast agent may have this influence directly as supplied, or in an at least partially diluted form, after being diluted by the medium in the body lumen (e.g., blood). For example, if measurements are indicative of the electrical resistivity of the medium in the lumen, and the contrast agent has a higher electrical resistivity than the blood, the measurements will tend to indicate an increase in electrical resistance. If, on the other hand, the dielectric contrast material is more conductive than blood, measurements will tend to indicate lowered resistance, increased current, and/or a lower voltage needed to pass a constant current. If the occlusion leaks (incomplete occlusion), the conductivity will return toward its initial value relatively quickly; e.g., insofar as the dielectric contrast agent will have a larger and/or more rapidly accessible pool of non-contrast fluid to mix with. For example, if the two compartments comprise the left atrium and its appendage, the measurements may return to pre-injection values after less than 5 heart beats, and sometimes even after less than three. If the occlusion is close to perfect, the conductivity will return to pre-injection values very slowly, if at all (albeit if not at all, they may return toward pre-injection values to a detectable degree). For example, if the two lumens are the left atrium and its appendage, the measurements may return to pre-injection values after more than 10 heart beats, or even after 20 heartbeats or more. In this context, it is worth noting that in some embodiments, in which the passageway is in the heart or leads into or from the heart, the change of the electrical measurements over time reflects at least in part beating of the heart. For example, if the occlusion is poor, contraction of the distal lumen may be accompanied with a briefly increased rate of dilution of the contrast agent.

According with the above: in some embodiments, estimating the occlusion degree includes classifying the electrical measurements according to a predetermined criterion. For example, the classifying may include identifying how long it takes to the electrical readings to return to their pre-injection values (or how long to return half or any other predetermined portion of the way towards the pre-injection values). A decay rate may be determined, optionally, based on such times). The identified time period or rate may then be compared to a threshold that may be determined empirically. For example, the classification may be so that if the measurements return to pre-injection values within 5 seconds or less, the occlusion is considered unsatisfactory. In some embodiments, there may be two or more thresholds, for example, 5 seconds and 20 seconds, so that if the measurements return to pre-injection values within a time period shorter than the first threshold the state of occlusion is unsatisfactory, if they return to pre-injection values after a time period longer than the second threshold, the occlusion degree is satisfactory, and if they return to pre-injection values after a time period shorter than the second threshold but longer than the first, the state of occlusion is intermediate.

Returning now to the electrical measurements themselves, these may include measurements of voltage between the electrode portion of the occlusive device and a reference electrode. The reference electrode may be, in some embodiments, attached to a body surface of the patient, e.g., a body-surface patch. Alternatively, the reference electrode may be on a catheter inside the patient’s body. For example, the reference electrode may be on a sheath used for delivering the occlusive device to the passageway it has to occlude. Optionally, the reference electrode is grounded (held at a stable potential, for example by grounding it to earth).

In some embodiments, the electrical measurements include measurements of electrical current injected to the electrode portion of the occlusive device. For example, the electrical measurements may include impedance measurements. The impedance may be defined by the proportion between the potential difference measured between the electrode portion of the device and the reference electrode, and current injected to the electrode portion of the occlusive device. In some embodiments, the current is constant, at least to a reasonable approximation, and in these embodiments, the voltage measurements may be indicative of the impedance.

In some embodiments, signal contributions from interfering sources are removed from electrical measurements to prepare the measurements for use in detecting a state of occlusion. Important sources of interfering signals include cyclic biological processes such as the respiratory cycle and the cardiac cycle.

In some embodiments of the present disclosure, signal contributions of one or both of these cycles to the electrical measurements are determined, and then removed, e.g., by subtraction, division, frequency filtering, and/or another method. In some embodiments, an iterative approach is used to characterize an interfering signal contribution so that it can be compensated for.

Determination of the signal contributions optionally uses assumptions about cycle frequencies. For example, the cardiac cycle typically exceeds about 45 beats per minute or greater, while breaths per minute is typically in a range of about 5-15. Frequency domain components in the measurements (e.g., as obtained by a Fourier transform of the measurements) can be matched to these assumptions. Doing this for the major cyclic components reduces the “energy” of their interfering signal contribution in the measurements. This potentially makes it easier to characterize the interfering signal contribution from the other major interfering signal contributor, and/or to characterize the decay signal of actual interest, since whatever model of the signal is used will not be required to fit measurement changes due to an irrelevant interfering process.

However: a typical perturbation decay signal occurs over the course of a several seconds. As a result, the breathing signal's frequency components can be somewhat close to (and hence, relatively difficult to distinguish from) the decay signal which is to be isolated. Removing it as just described may also distort (remove) some of the decay signal which is to be isolated. This may be true also with other methods of estimation which may be used additionally or alternatively to frequency component analysis. In short: the decay signal interferes with estimation of the cyclic noise sources, just as the cyclic noise sources interfere with estimation of the decay signal.

In some embodiments, this problem is potentially mitigated by a method of successive approximation. The decay signal is optionally modeled as non-repeating and “simple” (e.g., monotonic and approximately exponential decay). However, precise values of its parameters (e.g., decay constant and amplitude) may be obscured by any remaining noise from the interfering signal contributions, and/or distorted by the stage of initial signal processing based on cycle frequency assumptions as just described. Nevertheless, in some embodiments, best fit parameters for the estimated parametric form of the decay signal are matched to the remaining signal to derive a decay signal estimate.

This decay signal estimate may be only approximately representative of the actual decay signal; e.g., since some of the targeted signal may now be “missing”, e.g., taken away along with the respiratory cycle signal. However, the decay signal estimate may also, conversely, be viewed as relatively free of signal interference contributions, and in particular, contributions from the respiratory cycle.

Insofar as this is true, the decay signal estimate can be usefully subtracted from an earlier stage of processing (e.g., a stage after removal of the heartbeat cycle contribution, but before removal of the respiratory cycle contribution). Now the respiratory cycle’s interfering contribution to the measurements can be estimated again; optionally by the same or a different method; e.g., by using a signal averaging method synchronized to the phase of the respiratory cycle. That estimate is potentially more accurate than would have been made by the same processing before, since it now operates on a signal that is less “distorted” by the decay signal. Finally, the new (potentially more accurate) respiratory interfering contribution signal estimate can be removed, also from an earlier stage of signal processing (e.g., the post-cardiac cycle removal stage) that still has the decay signal relatively intact. Parameters of that decay signal can be estimated again, this time with potentially more accuracy.

This process is optionally iterated further, although the second estimate of the decay rate has been found to produce results robust enough to be useful. Iteration also optionally involves the cardiac cycle, however, separating cardiac cycle signal interference is expected to be less of a problem when distinguishing decay constants in the range of several seconds or more.

An aspect of some embodiments of the present invention relates to an apparatus configured to carry out a method as described above. For example, the apparatus may include a processor configured to access electrical measurements measured using an electrode portion of the occlusive device; estimate a state of occlusion based on the accessed electrical measurements; and output an indication of the estimated state of occlusion.

In some embodiments, the processor is also configured to control a display to present the outputted indication. The display may or may not be part of the apparatus.

In some embodiments, the apparatus includes an interface configured to provide the processor with a predetermined signal when the dielectric contrast agent is injected. For example, a physician injecting the dielectric contrast agent may simultaneously push a button in a user interface of the apparatus, and this push of the button may send to the processor the predetermined signal. The button may be pushed by the physician manually or by foot. The processor is configured, in some such embodiments, to use the predetermined signal in estimating the state of occlusion. For example, the processor may be configured to use the predetermined signal to distinguish between measurements made before and after the injection, start monitoring the measurements change over time, etc.

In some embodiments, the apparatus further includes a voltmeter, configured to measure a voltage between the electrode portion of the occlusive device, and the reference electrode. Measurements of this voltmeter may be included in the electrical measurements used by the processor for estimating the state of occlusion, for example, as described above. Similarly, in some embodiments, the apparatus may include an electrical current source, configured to inject electrical current to the electrode portion of the occlusive device. The processor may be configured to use measurements made during such injection of current to estimate the state of occlusion. In some embodiments, the apparatus also includes an amperemeter configured to measure electrical current injected to the electrode portion of the occlusive device. The processor may be configured to estimate the state of occlusion based on such current measurements, for example, by dividing the voltage measurements measured by the voltmeter by the current measurements for obtaining impedance values, and estimate the degree of the occlusion based on time evolution of the impedance after dielectric contrast is injected to the vicinity of the electrode portion of the occlusive device.

In some embodiments, the apparatus is provided together with an electrical interconnection device (which may also be referred to as a “pinbox”), configured to receive signal measurements from an occlusive device used as an electrode, and condition them (e.g., amplify and/or digitize them) to a form which can be accessed by the processor for further analysis.

In some embodiments, an occlusive device is provided as part of a kit comprising an electrical conductor configured to connect to the occlusive device, and transmit electrical signals therefrom to a measuring device outside the body while the occlusive device is positioned within the body. The wire may comprise an electrical connector suited to receive and/or be received by a matching connector on the occlusive device. In some embodiments, the electrical conductor is thin enough to be passed along a catheter along with the device, and long enough to extend outside the body. In some embodiments, the electrical conductor makes electrical contact with another element inside the body (for example, a strip or lining of the catheter), and the electrical signal is transmitted out of the body via that other element. In some embodiments, the conductor’s connector is configured to release upon a sufficient application of force (e.g., tugging force) to release the wire. In some embodiments, a portion of the conductor is permanently attached to the occlusive device, and optionally the conductor is configured to be broken at a position near to the permanent connection, e.g., by being weakest at that position. In some embodiments, an open-sided loop of the conductor passes distally to engage with the occlusive device, and then proximally again into the catheter. To release the occlusive device from the conductor after implantation, the conductor is drawn fully through its engagement with the occlusive device. Alternatively, a loop of the conductor is broken, e.g., by cutting it or upon sufficient pulling being exerted, and both sides of the broken loop are extracted.

In some embodiments, a kit is provided to convert a device into a useful electrode. In some embodiments, the kit comprises an elongated conductor with a securing clip on its distal end that can be released upon an actuating command movement initiated at a proximal end of the connector. In some embodiments, a terminal end of the elongated conductor is configured to be threaded through a hooked portion or aperture of the device, and is secured in place proximal to the device, e.g., within a deliver sheath or optionally leading back outside the body. To ensure electrical contact with the device, tension is maintained upon the loop during deployment. To release the device, one end of the elongated conductor is released, and the other end is pulled back to draw the elongated connector through and out of its looped connection with the device. In some embodiments, a stiffened elongated conductor is provided (e.g., stiffened similar to a catheter guidewire), which can be maintained in electrical contact with the device by pressing it forward in concert with forward movement of the device. The kit may be provided with guides to help maintain the stiffened elongated conductor in place, so that it is reliably guided to contact with a suitable portion of the device being implanted. Upon completion of its use, the conductor is withdrawn. Optionally, the conductor has a portion sized to make a friction fit with a portion of the device, e.g., a lumen of the device. The friction fit is set to be releasable upon deliberate exertion of force, but tight enough to withstand the normal manipulations of device deployment. The kit optionally includes one or more connectors suitable to adapt the elongated conductor to attach to a signal recording system. Optionally, the kit includes circuitry adapted to condition electrical signals recorded from the device (e.g., by amplification, offset, and/or digitization) to a format which is calibrated or otherwise made suitable for further processing.

An aspect of some embodiments of the present disclosure relates to methods of estimating a state of occlusion between two compartments of a body lumen. In some embodiments, the occlusion is obtained by deployment of an occlusive device to divide a body lumen. In some embodiments, the occlusion comprises dividing the body lumen into separate compartments on either side of the occlusive device. In some embodiments, the occlusion comprises closure of a portion of the body lumen into a separate compartment. In some embodiments, the occlusive device is a left atrium appendage occlusion (LAAO) device, deployed within a LAA, to convert the LAA from a portion of the left atrium into a separate, closed compartment. In some embodiments, the occlusive device is at least partially porous.

In some embodiments, the occlusion is intermittent, due to the movement of native or implanted valve leaflets of a heart valve positioned, e.g., between a heart atrium and a heart ventricle.

In some embodiments, the state of occlusion is determined based on electrical measurements (e.g., measurements of voltage, current, and/or impedance) made using the occlusive device as an electrode. A potential advantage of using the occlusive device itself as a measurement device stems from the occlusive device being configured, by its nature, to be left in place once a sufficient state of occlusion is confirmed. There is, accordingly, no requirement to introduce another measurement device to confirm a state of occlusion, which could either mean leaving an extra device behind, or somehow finding a way to extract a measurement device from a compartment which has already been occluded, potentially disrupting the occlusion in the process.

Additionally or alternatively, in some embodiments, occlusion is indicated by impedance changes measured using another device, for example, body surface electrodes, between which there is an electrical pathway including the body lumen targeted for occlusion.

In some embodiments, the state of occlusion is determined based on electrical measurements measured in the vicinity of the occlusive device, to which said vicinity has been supplied (for example, by injection of new fluid and/or modification of fluid already present) a dielectric contrast agent. The dielectric contrast agent optionally comprises, for example: hypertonic saline, ice cold saline, hypotonic saline, warm saline, cold blood, warm blood, iodine-containing solution (e.g. Iodixanol), or another solution with dielectric properties distinct from the dielectric properties of the fluid (e.g., blood) that normally fills the compartments into which the body lumen is to be divided.

In some embodiments, the measurements comprise measurement of voltage, current, and/or impedance as a function of time, upon injection of the dielectric contrast agent.

Herein, the term “state of occlusion” denotes a measured and/or estimated status of closure of an aperture (opening) and/or passageway by a device which is deployed with the purpose of blocking said aperture and/or passageway. Equivalently, the state of occlusion may be referred to as a “degree of occlusion”. On either side of the site of attempted closure (whether or not it is complete) is a “compartment” of the lumenal space which is to be divided by a closure. When the occlusion is intermittent (e.g., as in the case of a valve), what may be measured is a transient state of occlusion, for example, to evaluate whether the transient state does nor does not prevent flow regurgitation.

The meaning of “full occlusion” is dependent on the procedure and/or device being used. In some embodiments, full occlusion is effectively a hermetic seal. In some embodiments, full occlusion means that a device stretches across the full area of an occluded opening, but does not necessarily indicate a hermetic seal—for example, a device may comprise a permeable net, mesh, membrane, or other structure. In such cases, full occlusion may be associated with a typical amount of permeability e.g., to fluid. “Partial occlusion” may nevertheless be qualitatively different than “full occlusion”. For a device deployed in a way which does not span the full area of an occluded opening, there is a gap, between the device and a wall of the opening that is more permissive to fluid communication than the normal permeability through the device. In some embodiments, this difference between permeability through the device and through the gap is enhanced, e.g., by choosing fluid perturbations which propagate with difficulty or even not at all across the nominally permeable portions of the device, and propagate much more effectively via the gap. For example, the fluid perturbation comprises the use of a particularly viscous contrast agent. In any case, a device portion which is permeable may nevertheless be almost completely disruptive to bulk flow within the prevailing conditions of pressure and/or viscosity, even if a diffusion component of fluid redistribution is present. When an occlusion state like a closed valve is transient but repeating, the occlusion measurements are optionally controlled to occur at specific cyclic phases, e.g., of a heartbeat cycle (and/or analysis is performed using occlusion measurement selected from those specific cyclic phases). Perturbations chosen for use in measuring transient occlusions may likewise be timed to specific cyclic phases, e.g., to occur just before or while a transient occlusion is expected to occur, so that leakage back across a regurgitating valve which may occur is more easily distinguished from flow through the valve in its open state.

The “state” or “degree” of occlusion can be expressed as a status of one or more of several different types. For example, the state of occlusion is optionally judged by classification to one or more of a plurality of categories. Optionally, the categories are task oriented; e.g., chosen based on whether or not success has occurred and/or corrective action is recommended, as for example: “sufficiently occluded” and “insufficiently occluded”. Optionally state of occlusion is ranked on a scale of rankings, continuous or discontinuous, e.g., a scale consisting of the integers 1-5. Optionally, state of occlusion is expressed in numeric terms, for example, a geometrical measure such as area (e.g., 50%, 80%, 90%, or 100% filling of an aperture and/or an absolute measure of area), and/or a functional measure such as flow (e.g., 80%, 90%, or 100% reduction in flow through an aperture, and/or an absolute measure of flow). Optionally, a property measurement which is causally correlated with state of occlusion is used directly as an expression of a state of occlusion; for example, a measurement of an electrical resistance between two compartments also optionally provides its state of occlusion as such, albeit the resistance is optionally provided together with a scale, categorization, or other indication of how it should be interpreted in terms of state of occlusion. State of occlusion is optionally estimated from signal properties of a measurement made over time; for example, signal stability, signal time course, signal frequency, signal decay rate, signal amplitude, signal phase, and/or other signal properties. In some embodiments, state of occlusion is expressed as a measure of the rate of perturbation signal decay, for example, in terms of percent of remaining perturbation cleared per heartbeat Alternatively, percent per unit time (e.g., second) is selected, but percent per heartbeat has the potential advantage of tying the decay rate to the amount of mechanical pumping action exerted. Optionally, perturbation decay rate is expressed in terms of overall volume of blood passing through one of the compartments, e.g., percent change per milliliter of blood flow.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Dielectric Dilution-Based Deployment Verification

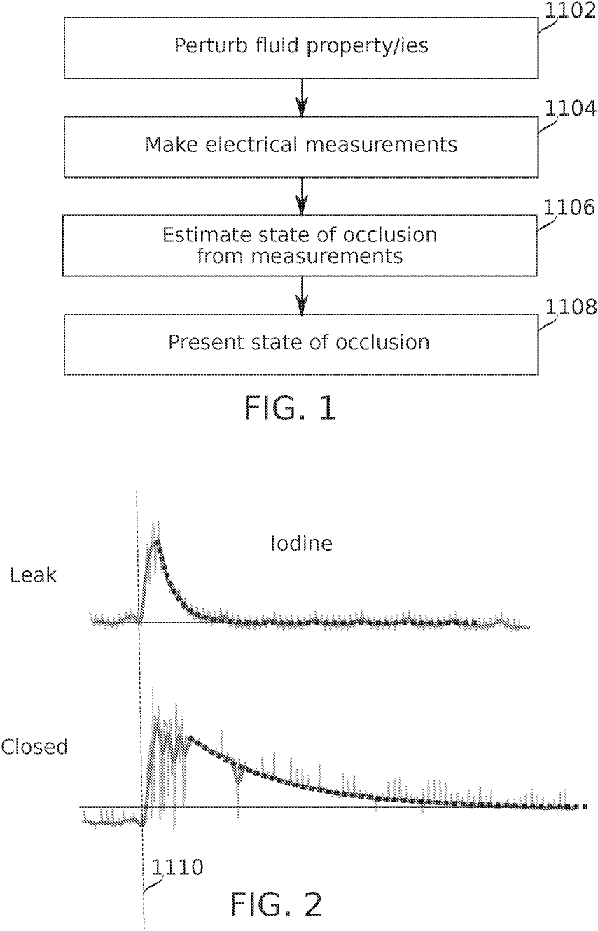

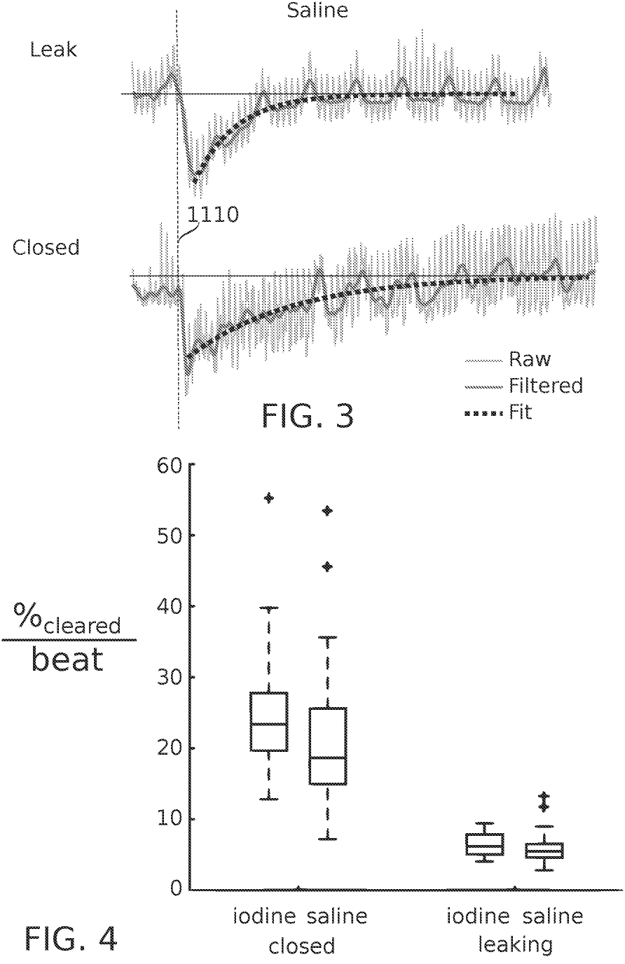

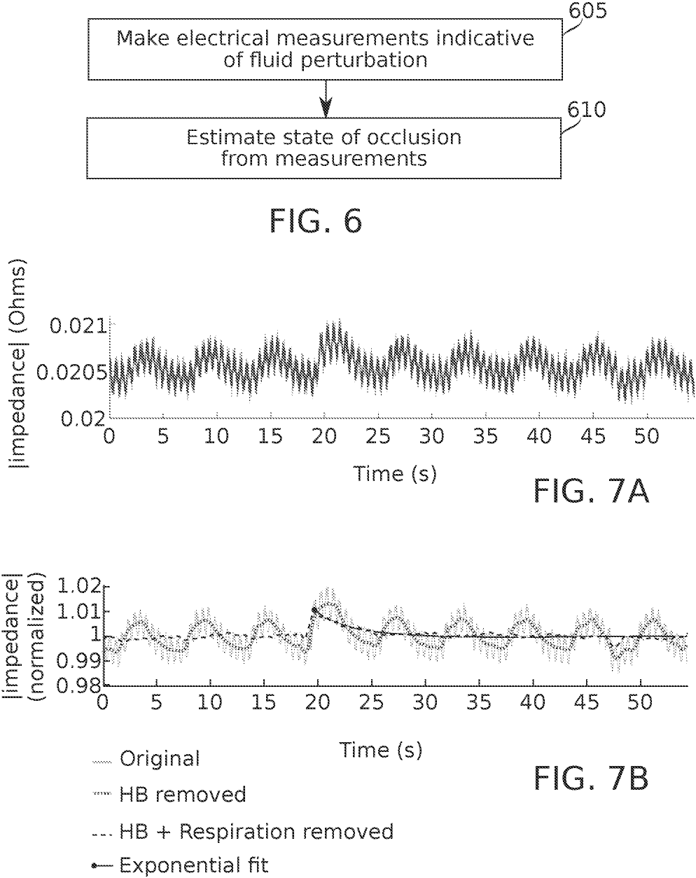

Reference is now made to FIG. 1, which is a flowchart schematically representing a method of monitoring state of occlusion between two fluid-filled compartments of a body lumen, according to some embodiments of the present disclosure. Reference is also made to FIG. 6, which is another flowchart schematically representing a method of monitoring state of occlusion between two fluid-filled compartments of a body lumen, according to some embodiments of the present disclosure. Reference is also made to FIG. 2, which shows example time courses of voltage measurements made using a Watchman™ LAA occlusive device as an electrode referenced (grounded) to a body surface pad electrode upon injection of Iodixanol solution, comparing leaking and closed deployments of an LAAO device, according to some embodiments of the present disclosure. Further reference is made to FIG. 3, which shows example time courses of voltage measurements made using a Watchman™ LAA occlusive device as an electrode referenced (grounded) to a body surface pad electrode upon injection of saline solution, comparing leaking and closed deployments of an LAAO device, according to some embodiments of the present disclosure.

In some embodiments, a correctly deployed implant device is an occlusive device that creates a characteristic level of closure that slows or prevents the exchange of fluid across it. Accordingly, the occlusive device affects the rate at which a contrast agent within the fluid will redistribute over time. A left atrial appendage occluder is an example of an occlusive device. Examples of other occlusive devices used to occlude passageways in the heart include devices for closure of an atrial septal defect, a patent (open) foramen ovale, and/or a ventricular septal defect. It should be noted that a body lumen, for purposes of the descriptions of FIG. 1, includes any two compartments in fluid communication with each other; for example, a left and right atrium together may comprise a “lumen”, insofar as they are interconnected by a patent foramen ovale.

In some embodiments, closure closes an opening in another organ, for example, an organ of the gastrointestinal tract (e.g., the stomach, for example to form a gastric sleeve).

At block 1102, in some embodiments, one or more fluid properties in the two compartments is perturbed. In some embodiments, the perturbed fluid properties comprise electrical properties of the fluid, e.g., a dielectric property such as resistance and/or impedance of the fluid. For example, a dielectric contrast agent is injected into one of the two compartments of the lumen divided by the occlusive device. Optionally, a non-injection perturbation is performed, for example, by use of warming, cooling, or induction of bubbles (e.g., ultrasonically).

A dielectric contrast agent comprises, for example: hypertonic saline, ice cold saline, hypotonic saline, warm saline, iodine containing solution (for example, Iodixanol), or another solution with dielectric properties distinct from the dielectric properties of the fluid (e.g., blood) that normally fills the compartments. In some embodiments, the contrast agent is injected as a bolus. In some embodiments, the contrast agent is injected continuously or nearly continuously.

In some embodiments, the injected dielectric contrast agent has a higher or lower viscosity than the normal-filling fluid. For example, a solution iodine contrast agent (e.g., Visiopaque™ 320, comprising about 320 mg I/ml) may have a viscosity (e.g., at 20°) of about 26.6 mm²/sec, compared to about 1 mm²/sec for water/saline. A range of typical viscosities of blood (at about 37°) is, for example, about 2.8-3.8 mm²/sec. Use of a higher-viscosity material potentially slows or prevents contrast agent migration, potentially making it easier, in turn, to distinguish between faster and slower redistribution times.

Either of the two compartments is optionally used, in any combination of one or both compartments, for injecting (or otherwise perturbing) from, injecting (or otherwise perturbing) to, and/or electrically measuring in.

In some embodiments, injection from one compartment to the other is optionally achieved by using relative high pressure injection (e.g., about 1200 psi) to inject the solution across a porous membrane of the occlusive device, needle injection (e.g., using a needle inserted across the occlusive device from one chamber to the other), and/or port injection (e.g., using a port of the occlusive device, optionally a one-way valved port). In the case of an LAAO device, for example, the delivery catheter is optionally pressed against a fabric or other porous membrane of the deployed occlusive device from the side of the atrium, and contrast agent is forced across the membrane towards the appendage. Contrast material injected under pressure across a membrane, once across the membrane, may return to the other side primarily by slower diffusion, and/or by flow (e.g., in case of a sealing leak). The amount of injected contrast agent fluid is, for example, about 10-20 ml. The injection is optionally continuous, or timed to coincide with phases of the heartbeat cycle, e.g., to a first 50%, a final 50%, and/or portions of either the first or final 50% of the heartbeat cycle. Optionally, separate injections are pulsatile with a repetition cycle that is longer than the cardiac cycle.

At block 1104 (FIG. 1) and block 605 (FIG. 6), in some embodiments, electrical measurements are made using a configuration of electrodes positioned in one or both of the compartments between which sealing is being assessed, and which are used as sensors sensitive to signal changes which a fluid perturbation (e.g., the fluid perturbation of block 1102) elicits.

In some embodiments, electrodes of the configuration comprise one or more electrodes within the mutually occluded compartments, or in another intra-body location. In some embodiments, the electrodes of the configuration comprise one or more body surface electrodes. For example, a body surface electrode is optionally used as a reference electrode relative to an intra-body electrode. Additionally or alternatively, body surface electrodes are used referenced to each other. The commonality of these configurations is that the region of perturbed fluid comprises part of the pathways along which electrical current flows between measurement electrodes. Changes in electrical properties in this part in turn affect the measurements. Using electrodes closer to (e.g., within) the perturbed fluid potentially improves the signal-to-noise ratio with which such changes are detected. Sensitivity (e.g., of body surface electrodes) to perturbation-elicited changes is optionally enhanced, e.g., by applying differential analysis to signals measured between pairs of electrodes positioned in different locations, frequency filtering tuned to the timecourse of the expected signal (e.g., several seconds), and/or use of perturbations which provide stronger dielectric contrast (larger boluses, higher concentrations). Some more particular examples of configurations of electrodes are further discussed hereinbelow.

In some embodiments, the electrical measurements comprise at least two measurements which show a change between them due to the perturbation. Herein, the measurements are said to be measurements of an electrical signal. The electrical signal comprises changes (e.g., of a measured current, voltage, and/or impedance) as a result of the fluid perturbation and processes such as diffusion, mixing, and/or jetting which interact with the fluid perturbation.

In some embodiments, a time-course of dielectric property changes is measured (e.g., by monitoring electrical measurements) over a period of within, e.g., the next 5-60 seconds after a bolus of dielectric contrast agent is delivered. In the change over time of data presented in FIGS. 2-3, the onset 1110 of contrast agent injection is followed by a rise (iodine containing agent, FIG. 2) or fall (saline, FIG. 3) in measured voltage which ends when injection ends. Afterward, measured voltage returns toward baseline with greater or lesser speed, depending on the rate of contrast agent redistribution. In the raw (light-colored) data, high-frequency spikes correspond to changes in voltage (used as an indication of local dielectric properties) due to heart beating, one spike per heartbeat. The spikes can be filtered out (darker-colored), emphasizing the long-term trend. For analysis, the data are optionally fit by a decaying exponential, for example of the form:

$$\frac{{\%}_{c l e a r e d}}{b e a t} = 100 \left(1 - {e}^{- \frac{H . R .}{{k}_{2}}}\right)$$

Where H.R. is the heart rate, and k₂ corresponds to a time constant with which impedance returns to baseline. Closure corresponds to a value of k₂ larger than about, for example, 10 seconds, or larger than about 20 seconds.

Brief reference is now made to FIG. 4, which summarizes experimental results of closed occlusion vs. leaking occlusion time-course measurements for a population of LAAO closure trials (in pig hearts), according to some embodiments of the present disclosure.

Clearance of a percentage of remaining contrast agent per beat

$$\left(e , g . , \frac{{\%}_{c l e a r e d}}{b e a t}\right)$$

is a way beat of expressing the rate of contrast agent redistribution after injection. 109 trials were performed in total (divided among the different conditions). The ranges of population quartiles are shown by the dotted bars (top and bottom quartiles) and boxes (middle two quartiles). Outlier examples (beyond the nominal top quartile) are shown as marks. A clearance time of below about 10 seconds (with either iodine or saline) also corresponded to a leaking device deployment. Iodine clearance was slightly slower than saline clearance.

With continuing reference to FIG. 1: additionally or alternatively, in some embodiments, dielectric contrast agent is delivered continuously and/or in a plurality of pulses (e.g., in several small amounts optionally timed to the heartbeat cycle and/or continuously). Blood heating using a heating element is an example of a perturbation which effectively creates a dielectric contrast agent out of existing fluid (“tagging” it), and can also be delivered continuously and/or in a plurality of pulses. Ultrasonic generation of bubbles is another tagging method capable of being used continuously and/or repeatedly. The continuing delivery of perturbations potentially results in an accumulating dielectric property change (in the contrast agent receiving chamber) once closure is achieved, while before that the dielectric change is prevented from accumulating (or from accumulating as much) by more rapid dilution of the dielectric contrast agent into a larger pool of surrounding and/or interconnected fluid. Additionally or alternatively, dielectric property change in a non-receiving chamber is reduced and/or delayed as closure improves, due to less dielectric contrast agent passing into it, and/or passing in less quickly.

Detection of dielectric change is performed, in some embodiments, using an electrode positioned in a chamber referred to herein as the detection chamber. The detection chamber can be the injected-to chamber; or a spread-to chamber, to which perturbation (e.g., injected or “tagged” contrast material) potentially leaks-to after injection. In some embodiments there may be more than one detection chamber, in which case one may be a spread-to chamber and another an injected-to chamber. The reference (ground) electrode is optionally, for example, another intra-body electrode (optionally, one located within the detection chamber, or on an opposite side of the occlusive device, e.g., in the injected-to chamber), or a body surface electrode.

In some embodiments (e.g., embodiments where the closure device is an LAAO device), the electrode used for measuring the electrical measurements is optionally an electrode of a sheath used to position and deploy the LAAO device, an electrode mounted on the LAAO device, and/or an electrode portion of the LAAO device itself. Optionally, the reference electrode is another electrode selected from among any of these electrode types. Optionally, the reference electrode is a body surface electrode attached, e.g., to the patient’s leg. In some embodiments, the reference electrode may be a body surface electrode attached to the patient's chest, or body side.

At block 1106 and block 610, in some embodiments, the measurements are used to estimate a state of occlusion. Optionally, a time course of dielectric property changes is classified, and the classification used as the estimated state of occlusion. The classification, in some embodiments, is performed automatically, e.g., by a suitably programmed computer processor. In some embodiments, the classification is between two categories: “leaky” and “non-leaky” (and optionally on a scale between the two). The criteria of classification are selected depending on the type of leak which is to be detected; e.g., some leaking is potentially permissible through and/or around an implant, as long as the rate of leakage is maintained below some threshold. For example, an LAAO device, in some embodiments, comprises a fabric or other porous membrane that allows contrast agent to pass through it, but at a rate which is distinguishably slower than leakage through tissue-device gaps.

In some embodiments, for example, a well-closed LAAO device creates an at least partial seal wherein return to a baseline level after an initial impedance change from baseline to its maximum amplitude (post-injection), takes about 40 seconds or more. The return is not necessary to exactly the pre-injection amplitude. It may return, for example, to a level near baseline within 10% of the maximum amplitude. For a poorly-closed LAAO device, return-to-baseline or rate constant times will be faster (e.g., at least twice faster) than for a well-closed LAAO device. For example, the return to baseline or near-baseline takes, less than about 20 seconds, or less than about 10 seconds. Optionally, a certain time to return to baseline (e.g., 20 seconds) is selected as a threshold cutoff between “leaky” and “non-leaky” deployments. Optionally, a return to baseline/near baseline faster than some lower threshold (e.g., 10 seconds) is classified simply “leaky”; a return to baseline/near baseline slower than some higher threshold (e.g., 40 seconds) is classified simply “non-leaky” and anything in-between is classified as intermediately leaky, optionally according to how close it is to the leaky or non-leaky threshold.

In some embodiments, additionally or alternatively to time to return to baseline, a different measure may be used, for example, time to return half the distance from the maximum to the baseline, or a rate constant, expressed, for example, in terms of % return per heartbeat, or another expression that describes the rate of returning to baseline. The inventors have found that the return to baseline is typically exponential, so every measure of exponential decay, such as half-life time or percentage change per time unit may be similarly useful for estimating the state of occlusion. The rate constant is optionally determined from a measurement time course which has been noise and/or frequency filtered, e.g., to remove transient effects due to heartbeat and/or breathing movements. However, there may also be information within the period of a heartbeat or other motion; for example, jets of fluid which occur during heart contraction potentially result in measurements that provide indications of leak existence, magnitude, and/or location.

In some embodiments, dilution and/or leakage of the contrast agent (as measured by electrical measurements) is classified by comparison to a baseline established by a model of how fluid crosses the closure device, how fluid is otherwise exchanged within the chambers (e.g., exchange through natural or artificial perfusion) and/or by example measurements obtained in tests of the device.

In some embodiments, the baseline model includes calculation based on an anticipated equilibrium or near-equilibrium distribution of dielectric contrast agent. For example, in some embodiments, dielectric contrast agent is modelled to be injected into one of two compartments, neither of which is actively perfused. In such embodiments, the baseline is based on a distribution of the dielectric contrast agent in, e.g., equal concentrations in either compartment. Additionally or alternatively, a target level of contrast agent concentration to be maintained in event of successful closure is modeled, e.g., a concentration twice that expected in the case of failed closure, when the closure is expected to cut the combined volume of the two compartments in half.

In some embodiments, the compartments are perfused relatively slowly compared to the rate of mixing/diffusion, and the model models concentration as the result of a dynamic balance of perfusion and of fluid exchange between compartments.

In an example embodiment, dielectric property change is measured from within the LAA, and on the side of an LAAO device within the LAA to which contrast agent is injected. The measuring electrode is optionally the LAAO device itself or a portion thereof. It is noted that the measuring electrode need not be affected only by the contents of the contrast agent-receiving chamber (the LAA in this example). For example, as the contrast agent leaks across it, the pumping action of the heart quickly dilutes and/or clears the leaking contrast agent from the other side, making it effectively “constant” (e.g., when looked at using low frequencies). Even if the fluid pools on either side of the occlusion were static (not flushed out), the redistribution of contrast agent potentially still tends to lower the average concentration of contrast agent detected by an electrode.

Conversely, an electrode located on the “leaked to” side (e.g., the atrium) is potentially capable of detecting the inverse signal (i.e. the contrast agent leaking into it). In the heart, for example, there may be a transient signal occurring with the frequency of the heart rate, as fluid is ejected from the LAA and/or accumulates in the left atrium during one period of the heartbeat, and is diluted and/or purged from the atrium during another period of the heartbeat. The signal may be larger and/or faster when there is a leak, in comparison to when there is no leak. The signal may be weak (e.g., below a threshold) or non-existent (e.g., smaller than the limits of detectability) when there is no significant leak. The threshold may be predetermined; for example, empirically or from model calculations.

Optionally, a plurality of electrodes are placed in the vicinity of the device-closed aperture between two compartments of a divided body lumen; for example, distributed along a straight, circular, or lasso-shaped electrode probe. Each such electrode potentially detects a change in its measurement value (e.g., a transient change due to heartbeat motion) influenced by its relative proximity to the leak. Accordingly, there are potentially differences in measurements among the plurality of electrodes. In some embodiments, differences are used to increase sensitivity of leak detection (e.g., to remove common background changes), and/or to help localize leaks to positions nearest electrodes that detect the largest and/or fastest change. Optionally, an estimate of leak position is part of the leak classification; for example, a circumferential quadrant (or other circumferential section) of the leak position is estimated.

Additionally or alternatively, “reverse-filling” is detected using a plurality of electrodes: for example, electrodes within the contrast agent-filled chamber detect inflow of the non-contrast (normal) fluid (e.g., blood) in bursts of higher amplitude and/or faster time course when they are positioned nearer to the leak that allows the inflow.

Optionally, correlations and/or differences among measurements made by different pairs of electrodes are used to help enhance and/or characterize a portion of the electrical signal due to the fluid perturbation and the subsequent evolution of electrical properties that the fluid perturbation elicits. For example, detection of “jets” of fluid near a leak is optionally improved by comparing measurements from electrodes near the jet, and electrodes further from the jet. A time-coordinated drop in perturbed fluid concentration (amount) in one compartment and rise in perturbed fluid in the other is an example of correlation information which is optionally used to enhance sensitivity to a state of occlusion.

At block 1108, in some embodiments, an indication of the state of occlusion is presented (e.g., displayed on a computer display) to an operator. The presentation optionally comprises presentation of a binary classification (e.g., leaky/non-leaky) and/or a scaled classification (e.g., a degree of a severity of leakage between leaky and non-leaky). The presentation optionally comprises an estimate of leak position. Optionally, classification is skipped; for example, the raw impedance time course of an electrode is presented. Interpretation of the raw time course is optionally left to the experience of an operator, and/or there may be indications on the time course such as suggested thresholds for leak/non leak times, and/or estimations of waveform properties (such as overall decay rates). Presentation, in some embodiments, uses the user interface of a computer, and takes the form of, e.g., an image, text, color (e.g., warning color), sound, and/or another sensory stimulus.

### Apparatus for Occlusion Detection

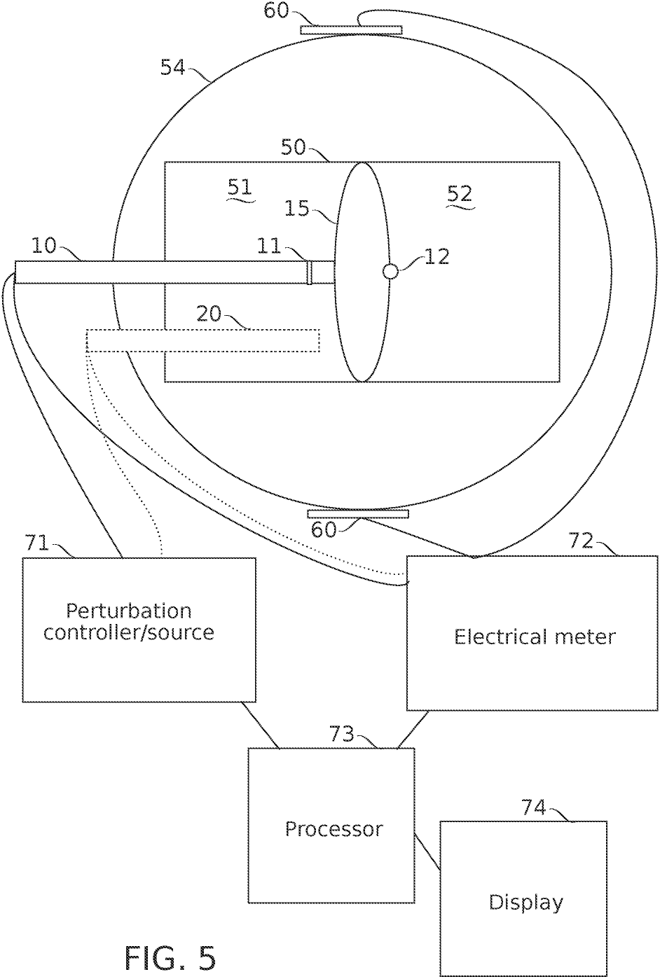

Reference is now made to FIG. 5, which schematically represents an apparatus for estimating a state of occlusion of an occlusive device 15, according to some embodiments of the present disclosure. It may be appreciated from the descriptions herein that embodiments of the apparatus of FIG. 5 may be used to implement methods described herein for estimating and/or monitoring degree of occlusion (for example, as described in reference to FIG. 1).

Indicated elements which are part of a human body (and not the apparatus) include body 54, lumenal space 50, and compartments 51, 52 into which lumenal space 50 is divided by occlusive device 15. Furthermore, compartment 51 represents a proximal lumen (on the side of the delivery device 10 for the occlusive device 15), and compartment 52 represents a distal lumen (on the side opposite).

It may be appreciated from the descriptions herein that embodiments of the apparatus of FIG. 5 are optionally provided with different configurations of electrodes. Shown in FIG. 5 are electrode portion 12 of occlusive device 15, an electrode 11 attached to a delivery device 10, and body surface electrodes 60. One or more additional/alternative electrodes may be provided, e.g., on auxiliary probe 20 (of which there may be none, one, or a plurality).

Electrical meter 72 receives electrical measurements made using the available set of electrodes 12, 11, 60. Optionally, operation and/or configuration of electrical meter 72 are under control of processor 73; for example to allow dynamic configuration of gain, offset, sample rate, and/or gating to produced readings within ranges known to the processor and/or suitable for producing useful measurements. Processor 73, in some embodiments, performs estimating of the state of occlusion, based on the electrical measurements, and optionally an indication of the state of occlusion is presented on display 74.

It may be appreciated from the descriptions herein that embodiments of the apparatus of FIG. 5 are optionally provided with different means to induce fluid perturbation. Perturbation controller/source 71, in some embodiments, is used to perform fluid perturbations, e.g., perturbations of one or more of the types described herein, for example, by injection, heating, cooling, induction of cavitations, or another fluid perturbation method. Perturbation may be performed through delivery device 10 itself, and/or through an auxiliary probe 20. Either or both of compartments 51, 52 may receive the direct effects of the fluid perturbation.

### Examples

Various embodiments and aspects of the present disclosure as delineated hereinabove and as claimed in the claims section below find experimental support in the following examples. It should be understood that embodiments of the present disclosure are not limited to just those of the particular examples, which are provided as illustrations of principles by which features of the claims may be carried out, and/or examples of results obtained using those principles.

### Examples of Signal Processing to Calculate Clearance Per Beat

Reference is now made to FIGS. 7A-7B, which schematically illustrate a method of isolating from a cardiac recording impedance changes occurring after introduction of a dielectric contrast agent, according to some embodiments of the present disclosure.

A decay time constant k₂ (alternatively expressed herein as 1 /τ) characterizes the rate of impedance change (e.g. due to dilution) after the introduction of a dielectric contrast agent affecting the impedance of a medium; for example, by injection and/or in situ manipulations such as heating or cooling. The time constant k₂ may be isolated from an intracardiac recording of impedance by a process that rejects (e.g., filters out) signals with frequencies corresponding to the cardiac and breathing frequencies, then fits the residual (i.e., the filtered recordings, or the recordings remaining after the rejection) to an analytic function such as an exponential decay function.

FIG. 7A represents a starting recording of impedance. Impedance is represented as an absolute value of the measured impedance (in some embodiments, measured at 10-30 kHz). In this example, impedance is measured between a measurement electrode (which may be itself an LAA occlusion device being implanted, and/or attached thereto) and a body surface electrode

$$\left({z}_{0} = \left|{Z}_{W M \to B S}\right|\right) \text{.}$$

Data to be processed comprise, for example, a period from about 10-30 s before an event that changes impedance (20 seconds are shown in FIGS. 7A-7B) until the exponential has returned close to the baseline value.

Dielectric contrast agent was introduced into the LAA relatively rapidly compared to the dilution time constant; e.g., over about 1-3 heartbeats. Spillage into the LA during injection would have been quickly cleared by normal heart blood flow,

FIG. 7B represents stages of isolation of the dielectric contrast decay signal, and curve fitting with different corresponding traces. Without isolation, the dielectric contrast decay signal is potentially confounded by simultaneously recorded impedance signals; in particular, signals due to heart and/or respiratory motions. Isolation is performed, in some embodiments, by estimating these confounding signals and subtracting them from the original signal. It should be understood that this general strategy may be carried out in various ways; the examples here give specific implementations. Furthermore, isolation by subtraction of estimated confounding signals is not necessarily the only strategy for estimating the time constant of the dielectric contrast decay signal, although this strategy provides the potential advantage-conferred by careful “cleaning” of the raw data-of a particularly accurate decay constant estimate. This strategy also confers a degree of self-validation: since it explicitly accounts for the major components of the raw signal, data and/or analysis errors (noise artifacts, for example) will be left as potentially prominent residual signals.

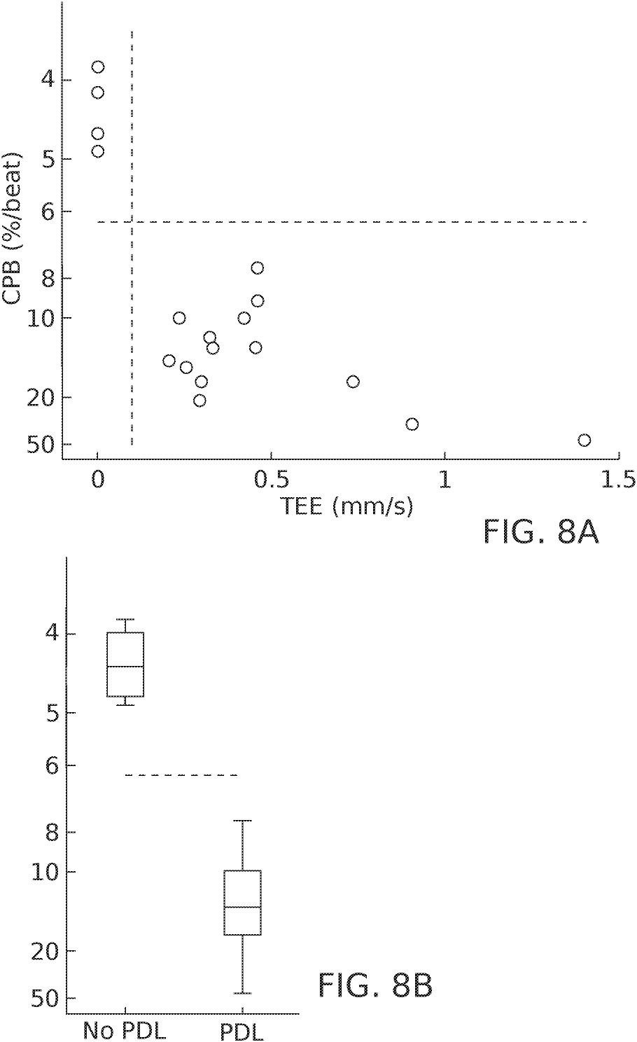

It is, however, not necessarily required that the time constant be estimated with high accuracy: the leak/non-leak difference may appear as a qualitative distinction between two groups comprising a wide separation in leak and non-leak time constants (e.g., as shown in FIGS. 8A-8B). Other strategies for time constant estimation may comprise, for example, using selected measurement values (e.g., peaks, valleys, and/or maximum slop magnitudes). A “most likely” leak/non-leak status may be estimated, for example, based on analysis of frequency power spectrum changes, magnitude of variance accounted for by exponential decays with different time courses, or another method.

In FIG. 7B, the original trace is in light gray, corresponding to the recording of FIG. 7A, normalized to values around 1, for example by division over a baseline value. In some embodiments, the normalization baseline z_{bl} is set to the mean of the low-pass filtered last 5 s of the signal. This normalization means the true baseline value is close to one, and subsequent curve fitting can be within a restricted range. The result is a signal vₐ, related to the original impedance amplitude according to

$${v}_{a} = \left|{z}_{0}\right| \text{} / \text{} {z}_{b l} .$$

Next, heart rate is estimated and removed to produce the HB removed trace (referred to below as z_{Lp}). In some embodiments, this comprises:
Heart-rate (HR) estimation: f_{HR} is optionally estimated by locating the peak frequency-domain component, for example at above 45 bpm. Other estimation methods may include, for example, measuring intervals between recurring features such as peaks, valleys, and/or regions of high slope magnitude. In some embodiments, the heart rate may be estimated based on output of other meters, such as a heart pulse monitor and/or ECG.
Cardiac frequency filtering: The heartbeat signal may be isolated by filtering; e.g.,
vₐ is optionally filtered with a 4^{th}-order Chebychev low-pass filter with a cut-off frequency at, for example, 10%-20% below the f_{HR}, to calculate a new signal v_{f}. In some embodiments, filtering is applied in the forward and reverse directions to ensure zero phase offset. Edge effects are optionally reduced using padding comprising, e.g., 15 s of the signal mean. Additionally or alternatively, the heartbeat signal may be estimated by another method, e.g., calculating the pattern of an average (or other “typical” heartbeat cycle from the raw data and repeating it (optionally, repeating with best-fit of time and/or amplitude scaling parameters).

From this result, breath rate is estimated and removed to produce the HB + Respiration removed trace. Two alternative methods of doing this are next described. Both use two successive exponential fits to isolate the breathing signal from the dilution/decay signal. The first method also includes an initial first approximation of the breathing signal.

The first method is optionally one or two stage. The output is referred to below as z₁ (if only the first of the two stages is performed), or z₂ (for the output of both stages). The two-stage approach potentially reduces the impact of the exponential on the estimate of the respiratory signal. Performing both stages can give significantly improved signal isolation.

In some embodiments, the first stage comprises:
Breath-rate (BR) estimation: f_{BR} is estimated by locating the peak frequency-domain component between 5 and 15 bpm.
Breath-rate signal estimation: z_{LP} is ensemble averaged at f_{BR} to create a breathing-only signal, z_{BR}, comprising a first pass at breathing signal estimation.
Removal of breath-rate interference: The breathing-only signal is subtracted from z_{LP} to create Z₁ = V_{f} - Z_{BR}

In some embodiments, the second stage comprises:
Candidate exponential fit identification: The best-fit of an exponential curve to z₁ is calculated, with parameters to reflect the start-time (t₀) (at the dot located in FIG. 7B at about 20 seconds), amplitude, and decay. This curve is zₑₓₚₗ. This step also identifies whether the contrast agent has increased (saline) or decreased (iodine) conductivity. This comprises a first pass at estimating the time course of impedance contrast decay.
Removal of exponential curve: The exponential is removed from the HR-filtered signal, to calculate z_{LP,2}=z_{LP}-zₑₓₚₗ.
Breath-rate signal estimation: z_{LP,2} is ensemble averaged at f_{BR} to create a breathing-only signal, z_{BR,2}, comprising a second pass at breathing signal estimation.
Removal of breath-rate interference: The breathing-only signal (second pass) is subtracted from z_{LP,2} to create TEST Z₂ = Z_{LP,2} - Z_{BR,2}

Finally, an exponential fit is performed on the HB + Respiration removed trace to produce the Exponential fit trace of FIG. 7B, for example as follows:

Exponential fit: Starting at the identified start time, tₛ=t₀+t_{d}, fit an exponential curve to minimize the norm

$$\left|{z}_{2} - \left(A {e}^{- \left(t - {t}_{s}\right) / k} 2 + B\right)\right|$$

for parameters A (amplitude), k₂, and B (baseline), and where t_{d}=1 s is a delay to avoid the distribution phase. This comprises a second pass at estimating the time course of impedance contrast decay.

The exponential fitting, in some embodiments, is implemented with the Nelder-Mead simplex algorithm. To avoid local minima, the curve fit is started from multiple values of the parameters, tₛ, A and B. Each individual curve fit output is then compared. The one with the lowest norm is selected.

Alternatively, in some embodiments, a first stage comprises:

Removal of first-pass exponential estimate: An exponential model is optionally based on five parameters (a,b,c,d,e) fit to v_{f}. Below, tₑ = t - e; the time offset to match the start of the exponential, and

$$v = \left\{\begin{matrix}a {e}^{- {t}_{e} /} {}^{b} & {\text{if t}}_{e} \geq 0 \\ a \left(1 - {t}_{e} / d\right) + c & \text{if d} < {t}_{e} < 0 \\ c & \text{if} {t}_{e} < d\end{matrix}\right\}$$

From multiple fitting start points, the best fit (lowest residual), vₘₗ is subtracted from the signal to obtain v_{fl} = v_{f} - vₘₗ. This comprises a first pass at estimating the time course of impedance contrast decay.
Breath-rate signal estimation: A breathing signal model is optionally created from v_{fl} in two stages. First, a best-fit sinusoid, v_{bl} is fit to the signal, with parameters of amplitude, frequency, and phase, from multiple starting points. Next, using the best-fit frequency and phase, three sinusoidal harmonics of the identified frequency are fit to the signal, to obtain the breath model, v_{b2}.
Removal of breath-rate interference: The breath model is subtracted from the filtered signal to obtain v_{f2} = v_{f}- v_{b2}.

Following this, in some embodiments, a second stage comprises:

Final exponential fit: A final exponential fit is obtained from v_{f2} over an interval starting t_{Δ} after the exponential peak (to ensure that only the elimination phase is fit). The resulting relationship between the fit and the data may be expressed as:

$$v = a {e}^{- {t}_{e} / τ} + c \text{for} {t}_{e} \geq t Δ$$

This comprises a second pass at estimating the time course of impedance contrast decay.

It should be understood that there is no particular limitation to the specifics of either of the above two methods of calculation. For example, dual- or greater-repetition estimation of respiration signal and/or impedance contrast signal decay may be implemented with other parameters, and/or in other ways.

For the calculated values of HR and k₂ (first method) or τ (second method, where τ substitutes for ⅟k2), clearance per beat (CPB) may then be calculated using, for example:

$$\frac{\text{\%cleared}}{\text{beat}} \text{=100} \left(1 - {\text{e}}^{- {\left({\text{HR×k}}_{\text{2}}\right)}^{- \text{1}}}\right)$$

### Example CPB Measurement Results

Reference is now made to FIGS. 8A-8B, which graph sealing test experimental results in dog heart, according to some embodiments of the present disclosure.

Data were collected for 109 seal-test events, including 82 using saline and 27 using iodine contrast agents to induce impedance changes. For each event, impedance data measured using an intracardiac electrode referenced to a body surface electrode were analyzed using the algorithm of FIGS. 7A-7B. From among these sealing test events, 18 were conducted at the same time as transesophageal echocardiography (TEE) measurements. CPB results from these are shown individually in FIG. 8A. The presence of peri-device leakage (PDL) was defined as corresponding to a non-zero Doppler signal (in mm/s) in the TEE measurement through a gap between device and heart of 5 mm wide or greater, with a Nyquist limit optionally set around 25 cm/s (a setting which can help emphasize slower flow). Using this definition, these cases were separated into “PDL” and “no PDL” categories (summarized statistically in FIG. 8B, and left/right of the dashed vertical line in FIG. 8A). By setting appropriate thresholds (horizontal dashed lines of FIGS. 8A and 8B) it was possible to correctly classify all tests using the CPB values (i.e. 100% sensitivity and specificity). The correlation between CPB and TEE values was r=0.914.

Based on these data, the hypothesis that CPB values correspond to presence or absence of PDL was tested for significance. The inverse hypothesis that CPB is independent of PDL yielded a repeated-measures t-test result of (p<10⁻³), allowing the inverse hypothesis to be rejected.

### Deployment Verification Using Closure-Induced Impedance Changes

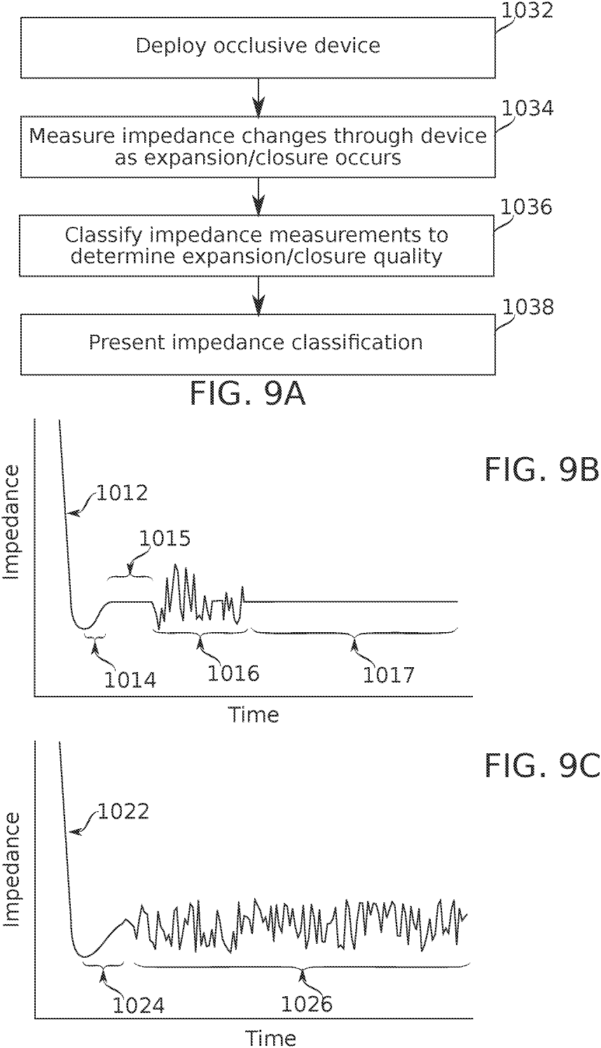

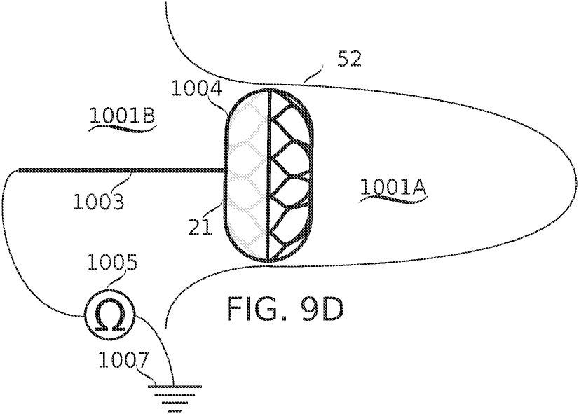

Reference is now made to FIG. 9A, which is a flowchart schematically representing a method of verifying implant positioning, according to some embodiments of the present disclosure. Reference is also made to FIGS. 9B-9C, which are schematic graphs of impedance over time for stable and unstable closure by an LAAO device, according to some embodiments of the present disclosure. Reference is also made to FIG. 9D, which schematically represents an electrical measurement configuration related to the measurements of FIGS. 9B-9C, according to some embodiments of the present disclosure.

FIG. 9D illustrates elements of the electrical configuration underlying the method/measurements of FIGS. 9A-9C. During closure by an LAAO device 21, two blood pools 1001A (e.g., inside the LAA 52), and 1001B (e.g., outside the LAA 52) become relatively electrically isolated from each other due to the interposition of metallic device 21 and the relatively insulating membrane 1004 (e.g., a fabric membrane) attached thereto. Device 1003 is attached to an electrical impedance measuring device, allowing an impedance 1005 to be measured, e.g., relative to the voltage of an electrode 1007 configured for use as a ground reference (e.g., an electrode attached to a leg body surface).

At block 1032 (FIG. 9A), in some embodiments, an occlusive device is deployed (e.g., LAAO device 21 is expanded to about the configuration shown in FIG. 9D). Measurement may be performed using the occlusive device itself as an electrode. Additionally or alternatively, in some embodiments, measurement is performed using body surface electrodes; the occluder device potentially modifies electrical paths between body surface electrodes differentially, based on whether it fully occludes the LAA, or only partially (and, more particularly, modifies the paths for impedance measured, e.g., at RF frequencies).

As the occlusive device expands, impedance is measured through an electrically conductive portion of the device at block 1034. Increasing size (e.g., increasing exposure of surface area of the occlusive device and/or volume through which the occlusive device extends), leads to a drop in impedance 1012, 1022 (FIGS. 9B, 9C) measured using the device itself as an electrode. Additionally or alternatively, in some embodiments, impedance changes are measured through body surface electrodes. Further expansion begins to create a closure, cutting off conductive pathways through blood, eventually resulting in a rebound in impedance, e.g., as shown during time periods 1014 and 1024. Contact with the heart wall itself may also contribute a component of impedance change. If the rebound 1014 is missing or attenuated in amplitude, it potentially indicates (e.g., is classified as indicating) that deployment seriously failed, i.e., it apparently has not taken place within a closely confined space as expected.

As expansion continues, closure is potentially formed, e.g., during period 1015. Potentially after interruption by one or more periods of reconfiguration/settling 1016 having relatively noisy impedance (e.g., a period with noise amplitude at least 2x, 4x, 5x, or 10x the amplitude of the background measurement noise), the impedance value settles to a stable value 1017, which provides an indication of apparently stable deployment.

In an alternative scenario, the period of post-expansion noise in the impedance 1026 does not settle to a quiet state, providing an indication that deployment may not be well-closing and/or stably anchored.

In some embodiments, observation of the timecourse of impedance (e.g., as displayed on a screen) is used to qualitatively estimate deployment status.

Additionally or alternatively: at optional block 1036, in some embodiments, a processor configured to analyze impedance operates to classify impedance measurements as they are taken. The processor is optionally configured to distinguish between and identify any two or more of the states described in relation to block 1034. For example:
A period 1012, 1022 of rapid impedance decline down to a minimum is identified as a period of device expansion.
A period 1014, 1024 of impedance rebound is identified as indicating that the device is expanding within the confines of an aperture of about the dimension of the device.
A subsequent period of unstable impedance 1016, 1026, or of only brief stability 1015 (e.g., stable for less than a heartbeat cycle, less than five seconds, or less than another brief period) is identified as indicating that device anchoring is itself unstable and/or incompletely closing. Periods of instability may be further characterized by correlation with the heartbeat phase; for example, device anchoring may loosen during a certain phase of heart contraction, at which noise occurs.
A period of longer stability 1017 (e.g., stable for at least several second, e.g., 5 or more seconds) is identified as indicating that the device anchoring is adequately stable and/or closing. Optionally, the device is deliberately wiggled and/or tugged at in order to test that it is firmly seated; partial loosening which results potentially interrupts the period of stability 1017 with a return to instability.

At optional block 1038, in some embodiments, the identification is presented as guidance for an operator. The guidance optionally comprises an indication which converts one or more of the features automatically identified in the impedance timecourse to an indication of device status; e.g., an indication corresponding to one or more of the identifications described in relation to periods 1012, 1022, 1014, 1024, 1015, and 1017.

In case that the occlusion device 21 fails to achieve a targeted level and/or stability of closure, the operator optionally takes a corrective action. For example, the operation may re-collapsing device 21 and attempt deployment again (e.g., at a slightly different position).

Optionally, a current phase of device 21 opening is estimated using the impedance drop phase 1012, 1021.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term "a compound" or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

1. A method of providing an estimated state of occlusion resulting from an occlusive implant positioned between two compartments of a lumenal space of a body of a patient, the method comprising:
accessing electrical measurements measured using an electrode portion of the occlusive implant;
estimating the state of occlusion based on the accessed electrical measurements; and
providing an indication of the estimated state of occlusion.

2. (canceled)

3. The method of claim 1, wherein the accessed electrical measurements comprise measurements made during a period following an injection of a dielectric contrast agent within at least one of the two compartments.

4. The method of claim 3, wherein the occlusive implant has a proximal side attached to a delivery device, and a distal side; and the dielectric contrast agent is injected to the compartment on the distal side of the occlusive implant.

5. The method of claim 4, wherein the dielectric contrast agent is pressed across a membrane of the occlusive implant into the compartment on the distal side of the occlusive deviceimplant.

6. The method of claim 1, wherein the state of occlusion is estimated based on a variation of the electrical measurements over time.

7. The method of claim 3, wherein the estimating comprises identifying, by a computer processor, a rate at which the electrical measurements return toward a baseline value after the injection; and
estimating the state of occlusion based on comparison of said rate to a predetermined threshold.

8-9. (canceled)

10. The method of claim claim 7, wherein the state of occlusion is estimated to be a satisfactory state of occlusion when the identified rate is slower than the predetermined threshold.

11. The method of claim 1, wherein the lumenal space comprises lumenal portions of a heart, and the state of occlusion is estimated based on changes in the electrical measurements driven at least in part by beating of the heart.

12. The method of claim 1, wherein the occlusive implant occludes an opening between a left atrial appendage and a remaining lumen of a heart left atrium.

13. The method of claim 1, wherein the occlusive deviceimplant occludes an opening between a left atrium and a right atrium of a heart.

14. The method of claim 1, wherein the occlusive implant occludes an opening between a left ventricle and right ventricle of a heart.

15. The method of claim 1, wherein the electrical measurements include measurements of voltage between the electrode portion of the occlusive implant, and a reference electrode.

16. The method of claim 15, wherein the reference electrode is attached to a body surface of the patient.

17. The method of claim 1, wherein the electrical measurements include measurements of electrical current injected to the electrode portion of the occlusive implant.

18. The method of claim 1, wherein the electrical measurements include impedance measurements.

19. The method of claim 18, wherein the impedance is a ratio between a voltage and current, wherein the voltage is a voltage measured between the electrode portion of the occlusive implant and a reference electrode; and the current is an electrical current injected to the electrode portion of the occlusive implant.

20. An apparatus for presenting an indication of a state of occlusion resulting from an occlusive deviceimplant positioned between two compartments of a lumenal space of a body of a patient, the apparatus comprising a processor configured to:
access electrical measurements measured using an electrode portion of the occlusive deviceimplant;
estimate a state of occlusion based on the accessed electrical measurements; and
present an indication of the estimated state of occlusion.

21. The apparatus of claim 20, further comprising:
an electrical current source, configured to inject electrical current to the electrode portion of the occlusive implant;
a voltmeter configured to measure alternating voltage between the electrode portion of the occlusive implant and a reference electrode attached to an outer surface of the patient; and
wherein the electrical measurements include measurements of voltage, alternating at a frequency, made when current of said frequency is injected from the electrical current source to the electrode portion of the occlusive implant.

22. The apparatus of claim 20, further comprising thea display controlled by the processor to present the indication of the estimated state of occlusion.

23. The apparatus of claim 20, further comprising an interface configured to provide the processor with a predetermined signal when a dielectric contrast agent is being injected on at least one side of the occlusive implant, and the processor is configured to use said predetermined signal in estimating the state of occlusion.

24. The apparatus of claim 20, wherein the processor is configured to estimate the state of occlusion based on a change of the electrical measurements over time.

25. The apparatus of claim 23, wherein the processor is configured to estimate the state of occlusion based on a change of the electrical measurements over time beginning upon receipt of the predetermined signal .

26. (canceled)

27. The apparatus of claim 20, wherein the processor is configured to classify the electrical measurements to obtain the estimated state of occlusion according to a time duration beginning upon receiving the predetermined signal, and ending upon returning of the electrical measurements to their values pre-injection.

28. The apparatus of claim 27, wherein the processor is configured to compare the time duration to a predetermined threshold, and classify the electrical measurements based on the comparison.

29-37. (canceled)
